(19) United States
(12) Patent Application Publication
(10) Pub. No.: US 2026/0234644 A1
(43) Pub. Date: Aug. 13, 2026

(54) SYSTEMS AND METHODS FOR SCALABLE TRANSFECTION REAGENT PREPARATION

(71) Applicant: CORNING INCORPORATED, CORNING, NY (US)

(72) Inventors: John Fitzpatrick, Woburn, MA (US); Vasiliy Nikolaevich Goral, Painted Post, NY (US)

(21) Appl. No.: 19/152,683

(22) PCT Filed: Jan. 16, 2024
(86) PCT No.: PCT/US2024/011574

§ 371 Date: Jul. 31, 2025

Related U.S. Application Data
(60) Provisional application No. 63/442,319, filed on Jan. 31, 2023.

(51) Int. Cl. C12N 15/64; B01F 33/30; C12M 1/00

(52) U.S. Cl. CPC C12N 15/64; B01F 33/30; C12M 29/10; C12M 43/00

(57) ABSTRACT

Methods and systems are provided for scalable preparation of transfection reagent. A method includes providing a plurality of reagent components for mixing together to form the transfection reagent, and mixing the reagent components in a passive mixing module having an outlet for outputting a mixture of the plurality of reagent components. The method further includes providing a plurality of incubation containers fluidly connected to the outlet and configured for receiving and holding the mixture; filling one or more of the plurality of incubation containers with at least a portion the mixture from the outlet, where the filling occurs consecutively; and holding the at least a portion of the mixture in one or more of the plurality of incubation containers for a predetermined incubation time to form the transfection reagent.

### CROSS-REFERENCE TO RELATED APPLICATIONS

This application claims the benefit of priority under 35 U.S.C. § 119 of U.S. Provisional Application Ser. No. 63/442,319 filed on Jan. 31, 2023, the content of which is relied upon and incorporated herein by reference in its entirety.

### TECHNICAL FIELD

This disclosure generally relates to production of transfection reagent for biomanufacturing processes. In particular, the present disclosure relates to methods and systems for continuous, scalable production of transfection reagent to transfect cells in a culture vessel or bioreactor.

### BACKGROUND

Adeno-associated virus (AAV) is a commonly used viral vector for gene therapy. The most popular and straightforward method for producing AAV is polyethylenimine (PEI) mediated triple transfection of three plasmids into HEK293 cells. PEI is used for transfection (Hildinger, M. et al, 2007, Biotechnology Letter, 29, 1713-1721), and is highly charged cationic polymer that readily binds with highly anionic plasmid DNA. This allows the PEI to form ionic interactions with the phosphate backbones of DNA to form condensed PEI-DNA complex that can be transported into cells via endocytosis (Goldbey, W. T., et al., 1999, PNAS, 96, 5177-5181). Within the complex, the particular ratio between molar units of PEI nitrogen atoms to units of DNA phosphate atoms, known as the P/N ratio, has been found to corelate with enhanced transfection efficiencies (Guo, P., et al., 2012, J. Virol. Methods, 183, 139-146). Non-viral vector nucleic acid complexes for cell transfection, such as polyplexes, are typically prepared by bulk mixing and usually require an incubation time of 5 to 30 minutes to form stable DNA-transfection reagent complexes. Such an incubation time has been considered as a standard for conventional technologies. Previous work has noted the importance of DNA concentration in transient transfection (Huang, X., et all, 2013, J. Virol Methods, 193, 270-277). PEI-DNA particle size may have an effect on transfection efficiency, with larger particles more easily endocytosed by the cells. (Reed, S. E., et al., 2006, J. Virol. Methods, 138: 85-98). The factors that affect PEI-DNA particle size are the DNA concentration of the transfection solution, efficiency and speed of mixing PEI and DNA solutions, time the transfection solution is incubated before being added to the cells, and kinetics of the transfection mixture addition. In general, increasing the incubation time increases particle size at all DNA concentrations. Previous experiments have shown the diameters of PEI-DNA particles mixed for 15 min range from 200 to 500 nm depending on PEI/DNA ratio (Ahh, H. H., et al. 2008, Colloids and Surfaces a-Physicochemical and Engineering Aspects, 313, 116-120).

Gene therapy process development for adherent cells usually is performed at laboratory scale level starting from multi-well plates (e.g., 6-, 12-, or 24-well plates), adherent cell culture flasks (with surface areas of, e.g., 25-175 cm²) and moving into multilayer cell culture vessels (e.g., CellStack® or HYPERStack® by Corning®) for laboratory scale production. However, for large-scale production, the process needs to be further optimized in larger bioreactors (e.g., so-called process development bioreactors, which may have cell attachment surface areas of about 1-5 m², in some cases) and then scaled up to even larger bioreactors (e.g., having cell attachment surface areas of about 20-1000 m², sometimes called pilot or production reactors). As the size of the bioreactors is scaled up, all the reagent quantities and volumes are scaled up proportionately. Scalability of some of the process parameters that deal with physical movement of liquids—such as mixing kinetics or mixing uniformity—can only be addressed by appropriate system design at process development and pilot/production scale levels. For example, at laboratory scale user can mix two solutions in aseptic conditions almost instantaneously by pouring two samples into the vessel of larger volume. Such a step is impossible to directly scale up for production scale where 30 L samples need to be mixed. Therefore, process steps have to be adapted and “designed in” to achieve their scalability.

Attempts have been made to mix ingredients inside a bag (see WO 2018/208960 A1). This method has very limited scalability of key process parameters. As the volume of the reagents increases upon scale up from the process development to production scale, mixing kinetics and incubation time for formation of PEI-DNA nanoparticles cannot be maintained the same.

In addition, the incubation time of bulk mixing does not allow for production of DNA-transfection complexes in a continuous manner, thereby resulting in a discontinuous transfection process. Non-continuous transfection processes may be less reliable, less reproducible from batch-to-batch, and may also present scale-up challenges.

Examples of bulk mixing are reported in “Guide for DNA Transfection in iCELLis® 500 and iCELLis 500+ Bioreactors for Large Scale Gene Therapy Vector Manufacturing,” where two DNA transfection methods are provided. In such methods, the plasmids and the polyethyleneimine (PEI or PEIpro) are placed into two separate single-use bags. In one method, bulk mixing is performed using gravity to add the PEIpro to the DNA. In a second method, bulk mixing is performed using a peristaltic pump to add the PEIpro to the DNA. The iCELLis guide further recommends gently mixing the bag manually, which may present difficulty when handling large volumes. Thus, the bulk mixing process is non-continuous and prone to variability. Furthermore, Legmann et al., Transient Transfection at Large Scale for Clinical AAV9 Vector Manufacturing, B Poster Content as Presented at ISCT 2020 Virtual, May 2020, reports that the incubation time for complex formation by bulk mixing via iCELLis bioreactor is 25 minutes, thereby rendering the process discontinuous.

CN 104974933B reports a continuous mode transfection example, namely a device and method for long-term expression of recombinant protein in a continuous suspension bioreactor through multiple transient transfections. Although transfection is performed in a flow mode, the preparation of the plasmid/vector complex is not described but it is indicated that it requires an incubation time of at least 5 to 30 minutes to form stable DNA/transfection reagent complexes. Thus, the incubation time renders the process discontinuous.

WO 2018/208960A1 reports scalable methods of creating DNA and transfection reagent master mixes for transfecting cells. Methods include preparing a transfection master mix by introducing a DNA solution and a transfection reagent solution into a mixing container, and incubating the transfection master mix for an incubation period during which the transfection master mix is substantially still, the incubation period being between 5 to 180 minutes. However, the incubation time renders the process non-continuous.

Lu et al. (POLYPLEX SYNTHESIS BY “MICROFLUIDIC DRIFTING” BASED THREE-DIMENSIONAL HYDRODYNAMIC FOCUSING METHOD; ACS Nano. 2014 Jan. 28; 8(1): 332-339) reports preparing polyplexes in a continuous manner in microfluidic devices, namely synthesized DNA and/or polymer nanocomplexes using a 3D hydrodynamic focusing method. Lu et al. reports that the nanocomplexes prepared by the 3D focusing method have smaller size, slower aggregation rate, higher transfection efficiency, and induce similar cytotoxicity compared to the nanocomplexes prepared by bulk mixing methods. However, the throughput of such a microfluidic device reported by Lu et al. is low and requires significant numbering up to meet a volume of transfection solution required for manufacturing.

Consequently, a need exists within the bioprocessing industry for apparatuses and methods that enable scalable processes for preparation transfection reagent mix. In addition, there is a need for real-time continuous, scalable production of non-viral carrier nucleic acid particles to transfect cells cultured in a culture vessel or bioreactor.

### SUMMARY

In an aspect, a method for preparing a non-viral vector nucleic acid complex suspension is provided. The method comprises providing a passive mixing fluidic module comprising a plurality of inlets and an outlet; providing a transfection reagent solution to a first inlet of the plurality of inlets and a nucleic acid solution to a second inlet of the plurality of inlets; mixing the transfection reagent solution and the nucleic acid solution by flowing the solutions through the passive mixing fluidic module to create a resulting suspension; and outputting the resulting non-viral vector nucleic acid complex suspension from the outlet.

In an embodiment, the passive mixing fluidic module comprises a microfluidic mixing device. In an embodiment, the microfluidic mixing device comprises a plurality of mixing elements. In an embodiment, the plurality of mixing elements comprises heart-shaped mixing elements. In an embodiment, the microfluidic mixing device comprises a continuous microfluidic flow reactor. In an embodiment, the continuous microfluidic flow reactor is configured for scale-up from lab bench to industrial manufacturing.

In an embodiment, the transfection reagent solution comprises polyethylenimine (PEI), calcium phosphate, DEAE-Dextran, polyarginine, dendrimers, ionizable or cationic lipids, lipid-like lipidoids, or combinations thereof.

In an embodiment, the nucleic acid solution comprises plasmids.

In an embodiment, the method further comprises transfecting cells using real-time, continuous production of the non-viral vector nucleic acid complex suspension.

In an embodiment, the non-viral vector nucleic acid complex suspension is continuously delivered to a culture vessel.

In an embodiment, the culture vessel comprises a bioreactor. In an embodiment, the culture vessel comprises a perfusion bioreactor.

In an embodiment, the resulting suspension is delivered from the outlet to the culture vessel through sterile connectors.

In an embodiment, the culture vessel comprises cells to be transfected.

In an embodiment, the culture vessel comprises culture medium. In an embodiment, the culture medium comprises Fetal Bovine Serum (FBS).

In an embodiment, the resulting suspension is continuously delivered to a media source being perfused to the culture vessel.

In an embodiment, the microfluidic mixing device comprises a disposable mixing device. In an embodiment, the microfluidic mixing device is formed of a polymer material.

In an embodiment, the microfluidic mixing device comprises a reusable mixing device. In an embodiment, the microfluidic mixing device is formed of a glass material.

In an embodiment, a plurality of microfluidic mixing devices is arranged in series in the passive mixing fluidic module.

In an embodiment, the method is reproducible and consistent from batch to batch.

In an aspect, a system for preparing a non-viral vector nucleic acid complex suspension for use in transfection is provided. The system comprises a passive mixing fluidic module comprising a plurality of inlets, an outlet, and at least one microfluidic mixing device having a plurality of mixing elements; the passive mixing fluidic module having inputs of a transfection reagent solution and a nucleic acid solution; the passive mixing fluidic module having an output of a resulting non-viral vector nucleic acid complex suspension; and a culture vessel comprising cells to be transfected and culture medium and configured to receive continuous delivery of the resulting non-viral vector nucleic acid complex suspension to transfect the cells.

In an embodiment, the passive mixing fluidic module comprises a microfluidic mixing device. In an embodiment, the microfluidic mixing device comprises a plurality of mixing elements. In an embodiment, the plurality of mixing elements comprises heart-shaped mixing elements. In an embodiment, the microfluidic mixing device comprises a continuous microfluidic flow reactor. In an embodiment, the continuous microfluidic flow reactor is configured for scale-up from lab bench to industrial manufacturing.

In an embodiment, the culture vessel comprises a bioreactor. In an embodiment, the culture vessel comprises a perfusion bioreactor.

In an embodiment, the resulting suspension is delivered from the outlet to the culture vessel through sterile connectors.

In an embodiment, the microfluidic mixing device comprises a disposable mixing device. In an embodiment, the microfluidic mixing device is formed of a polymer material.

In an embodiment, the microfluidic mixing device comprises a reusable mixing device. In an embodiment, the microfluidic mixing device is formed of a glass material.

In an embodiment, the passive mixing fluidic module comprises a plurality of microfluidic mixing devices arranged in series.

In an embodiment, the transfection reagent solution comprises polyethylenimine (PEI), calcium phosphate, DEAE-Dextran, polyarginine, dendrimers, ionizable or cationic lipids, lipid-like lipidoids, or combinations thereof.

In an embodiment, the nucleic acid solution comprises plasmids.

In an embodiment, the culture medium comprises Fetal Bovine Serum (FBS).

In an embodiment, the system further comprises a cell culture medium source.

In an embodiment, the resulting suspension non-viral vector nucleic acid complex suspension is continuously delivered to the cell culture medium source being perfused to the culture vessel.

In an embodiment, the passive mixing fluidic module further comprises a T-junction or Y-junction. In an embodiment, the microfluidic mixing device comprises an in-line static mixer element. In an embodiment, the in-line static mixer element is connected after the T-junction or Y-junction. In an embodiment, the system further comprises peristaltic pumps. In an embodiment, tubing connected upstream the T-junction or Y-junction comprises double-Y tubing. In an embodiment, the system further comprises aseptic connectors. In an embodiment, the passive mixing fluidic module is configured for gamma irradiation treatment.

In other embodiments, a method for preparing a transfection reagent comprising: providing a plurality of reagent components for mixing together to form the transfection reagent; mixing the plurality of reagent components in a passive mixing module comprising a plurality of inlets and an outlet, the plurality of inlets being for receiving the plurality of reagent components and the outlet being for outputting a mixture of the plurality of reagent components; providing a plurality of incubation containers fluidly connected to the outlet and configured for receiving and holding the mixture; filling one or more of the plurality of incubation containers with at least a portion the mixture from the outlet, the filling of the one or more of the plurality of incubation containers occurring consecutively; and holding the at least a portion of the mixture in one or more of the plurality of incubation containers for a predetermined incubation time to form the transfection reagent.

### BRIEF DESCRIPTION OF THE DRAWINGS

FIG. 1 is a schematic of a system according to an embodiment of the disclosure.

FIG. 2 is a schematic of a system according to an embodiment of the disclosure.

FIG. 3 is a mixing fluidic module according to an embodiment of the disclosure.

FIG. 4 is a passive mixing module device according to an embodiment of the disclosure.

FIG. 5 is a graph showing the hydrodynamic diameter of polyplex particles determined by DLS as function of time once diluted in culture media according to an embodiment of the disclosure.

FIG. 6 is a graph showing polyplex size measurement and size evolution assessment during time for batch and low flow methods according to embodiments of the disclosure.

FIG. 7 is a chart showing potential zeta measurement of complexes formed with batch and low flow methods according to embodiments of the disclosure.

FIG. 8 shows images of cells adherent on mesh and expressing a similar level of GFP for batch and low flow methods according to embodiments of the disclosure.

FIG. 9 is a chart showing flow cytometry analysis for both batch and AFR low flow methods according to embodiments of the disclosure.

FIG. 10 shows charts depicting AAV2 quantification for both batch and AFR low flow methods according to embodiments of the disclosure.

FIG. 11 is a chart showing polyplex size measurement and size evolution of complexes formed with batch, low flow, and T-junction methods according to embodiments of the disclosure.

FIG. 12 is a chart showing potential zeta measurement of complexes formed with batch, low flow, and T-junction methods according to embodiments of the disclosure.

FIG. 13 shows charts depicting AAV2 quantification for both AFR low flow and T-junction methods according to embodiments of the disclosure.

FIG. 14 is a schematic of a system for scalable transfection reagent preparation, according to embodiments.

FIG. 15 is a schematic of a consumable set for the system in FIG. 14, according to embodiments.

FIG. 16A is a partial cross-section view of an in-line static mixer, according to embodiments.

FIG. 16B is a photograph of a mixer component from the mixer in FIG. 16A, according to embodiments.

### DETAILED DESCRIPTION

Methods described herein provide for preparation of non-viral vector nucleic acid complex suspension in a continuous and highly reproducible manner, providing consistency from batch to batch. Embodiments of the disclosure provide methods for transfecting cells using real-time continuous production of non-viral carrier nucleic acid particles, and more particularly a method using a specific setup for delivering the non-viral carrier nucleic complex to a culture vessel in a continuous mode. Embodiments of the present disclosure may be used to produce large scale recombinant protein synthesis by transient transfection and are particularly suited to the production of viral particles such as adeno-associated viruses (AAV) and lentiviruses. Embodiments of the present disclosure are directed to providing a mixing device to practice the transfection complex preparation. Methods and devices of the present disclosure allow for well-controlled transfection, reduce batch-to-batch variability, and allow seamless scale-up.

As described herein, a method for transfecting cells using real-time, continuous production of non-viral carrier nucleic acid particles is provided. The continuous production of non-viral carrier nucleic acid particles is obtained by intimately mixing the transfection agent(s) and the nucleic acid(s), such as plasmids, within a passive mixing fluidic module to create a non-viral carrier nucleic acid particle suspension.

In an embodiment, the passive mixing fluidic module or mixing device comprises a microfluidic mixing device. A nonlimiting example of a microfluidic mixing device comprises the Advanced-Flow™ reactor (AFR™) (available from Corning Incorporated, Corning, New York, USA) that provides seamless scale-up by design. If needed, multiple fluidic modules may be arranged, such as in series, to increase the residence time.

The non-viral carrier nucleic acid particle suspension created in the passive mixing fluidic module is continuously injected in a vessel containing the cells to be transfected without any incubation time, and the vessel is advantageously perfused by the culture medium. Unlike conventional methods such as batch or bulk mixing, the liquids according to methods described herein are always in motion and do not remain stationary, therefore allowing for a better homogeneity of the non-viral carrier nucleic acid particles suspension.

Because the passive mixing module is directly connected to the cell reactor, it allows for a controlled, constant, reproducible, and continuous transfection. Moreover, because the flow setup is directly connected to the reactor using sterile connectors, it does not require large size bags, handling, or hand-mixing, such as needed for conventional batch or bulk mixing protocols.

Furthermore, methods and systems described herein may also require less PEI than direct transfection methods, such as direct transfection methods reported in the literature which consist of injecting first the plasmids into the cell vessel and then injecting the PEI in a second step (Xie et al.; PEI/DNA formation affects transient gene expression in suspension Chinese hamster ovary cells via a one-step transfection process; Cytotechnology. 2013 March; 65(2): 263-271).

In an embodiment, the mixing device may be reusable. For example, in an embodiment, the mixing device may be formed of glass. When the mixing device, or mixing fluidic module, is made of glass, it can be readily cleaned by the CIP procedure and thus may be reused.

In an embodiment, the mixing device may be single-use or disposable. As a nonlimiting example, in an embodiment, the mixing device may be formed of plastic or a polymer material. A polymer or plastic-made mixing fluidic module may be ready to use with sterile connectors, would not require cleaning if used directly out of sterile packaging, and would be disposable.

Methods as described herein comprise intimately mixing together the transfection agent and the nucleic acid, e.g., plasmids, within a passive mixing fluidic module and continuously injecting the resulting suspension in the culture vessel. The non-viral vector nucleic acid complex suspension is discharged at the outlet of the passive mixing fluidic module and directly injected into the inlet of the vessel containing the cells to be transfected. During the injection, the culture medium may or may not be circulating through the vessel. In an embodiment, the non-viral vector/nucleic acid complex suspension may be injected directly in the media bottle when the vessel is under perfusion.

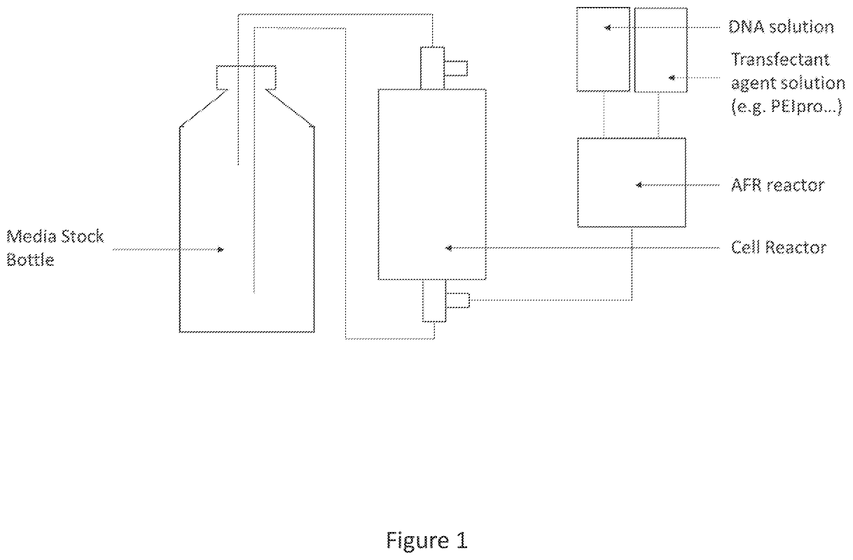

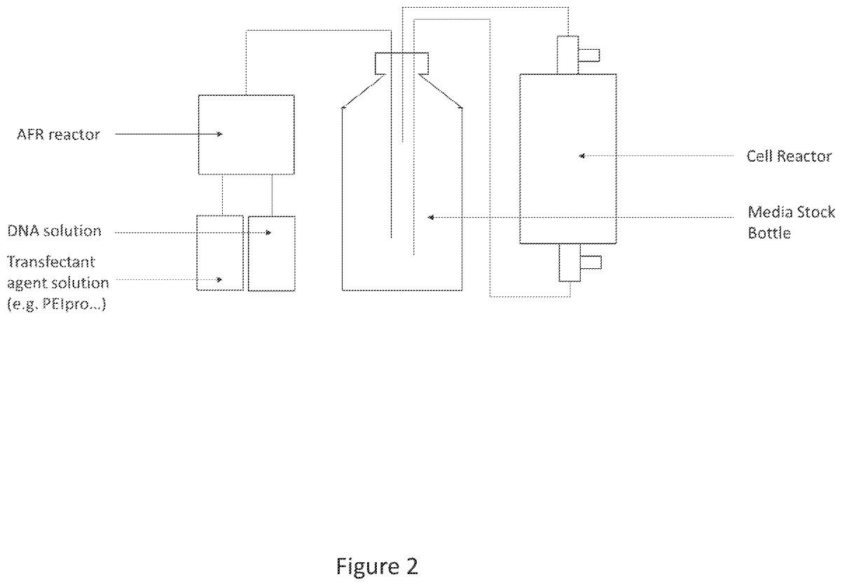

FIG. 1 shows an embodiment of a system where non-viral vector nucleic acid complex suspension is discharged at the outlet of the passive mixing fluidic module and directly injected into the inlet of the vessel containing the cells to be transfected. FIG. 2 shows an embodiment of a system where the non-viral vector/nucleic acid complex suspension may be injected directly in the media bottle when the vessel is under perfusion.

In FIG. 1 and FIG. 2, the system comprises a media source (e.g., cell culture media bottle), a nucleic acid solution (e.g., DNA solution, plasmids), a transfection agent solution (e.g., PEI), a passive mixing fluidic module (e.g., an AFR™ reactor), a cell culture vessel (e.g., a bioreactor, cell culture reactor, perfusion reactor).

During injection, the non-viral vector/nucleic acid complex becomes diluted by the culture medium. In an embodiment, the culture medium may comprise Fetal Bovine Serum (FBS). In an embodiment, the culture medium may comprise serum-free media.

### Passive Mixing Modules

The mixing module comprises at least two inlets and at least one outlet. In between the inlet and the outlet, the mixing device comprises mixing element(s). The mixing step is made by flowing the solution through the static mixing device that has no mobile elements as such mobile element could damage the nucleic acids by mechanical shear.

Supercoiled circular (SC) plasmid DNA is often subjected to fluid stress in large-scale manufacturing processes. Thus, methods and systems as described herein provide passive mixers, as no degradation of the nucleic acid is expected, unlike the dynamic mixers which could lead to shear-induced degradation.

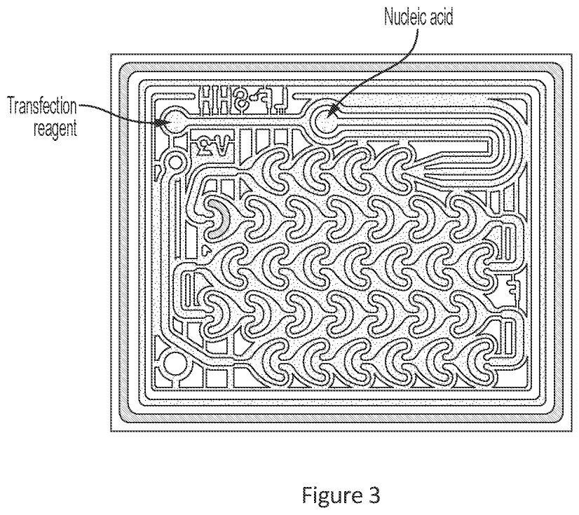

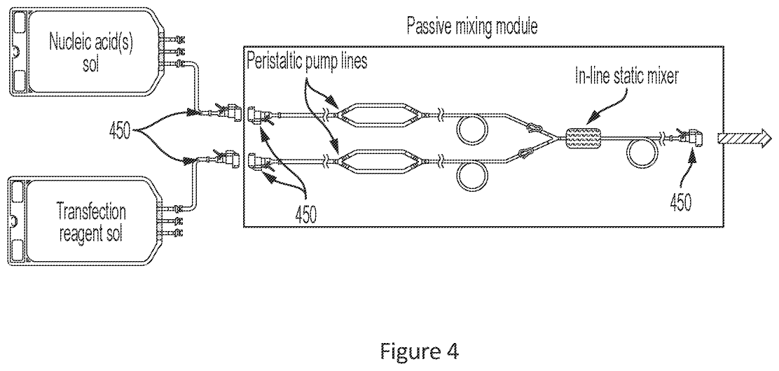

The passive mixing fluidic module may comprise a microfluidic mixing device. Such mixing elements of passive mixers may have various geometries. In an embodiment, the passive mixer may have heart-shaped mixing zones or heart-shaped mixing elements. The microfluidic mixing device may comprise a continuous microfluidic flow reactor. The continuous microfluidic flow reactor is configured for scale-up from lab bench to industrial manufacturing. In an embodiment, the mixing fluidic module or microfluidic mixing device is an advanced flow reactor (AFR™) fluidic module having heart-shaped mixing elements (available from Corning Incorporated, Corning, NY) (FIG. 3).

Although such types of passive mixers were originally designed for chemical synthesis, it has been surprisingly discovered that they are particularly well suited for the preparation of non-viral vector nucleic acid particles within a continuous mode. The inlets of such fluidic modules can be readily connected, such as by using aseptic connectors, to bags or other containers containing the plasmid(s) and the transfection reagent(s) or their precursors and the outlet of the said mixing module is easily connected to the culture vessel hosting the cell to be transfected.

Although a mixing fluidic module made of glass can be used, any other materials like plastics can be used provided that the surface of the material do not adsorb excessive amount of transfection reagents. The mixing fluidic module can be disposable and single use. As an example, a glass mixing module is particularly adapted to reuse, as a clean in place (CIP) protocol can be applied. CIP refers to a validated cleaning method involving automatic cleaning of interior surfaces of pipes, vessels, process equipment, and associated fittings without disassembly.

The method of the present disclosure is particularly useful for production of transfection complex, as the method may be used to meet the demand of large-scale bioreactor needs such as the Corning® Ascent® Fixed Bed Bioreactor (FBR) system (Corning Incorporated, Corning, NY). Table 1 shows the surface areas available to the cells to grow for different scales and the corresponding volumes of transfection mix required. Table 1 also shows the type of AFR™ modules (the passive mixing device) and the duration of the preparation of the complex varying from 1 minute to 20 minutes for 0.24 m² and 500 m² reactors, respectively.

TABLE 1
Surface Area with respect to AFR Systems and Parameters

| | Transfection mix | | |
|---|---|---|---|
| Ascent FBR | approximate | AFR | Preparation Duration |
| SA in m² | volume (L) | system | (max flow rate) min |
| 0.24 | 0.012 | Low Flow | 1 |
| 1 | 0.08 | Low Flow | 8 |
| 2.5 | 0.2 | Low Flow | 20 |
| 5 | 0.4 | G1 | 3 |
| 20 | 1.6 | G1 | 11 |
| 50 | 4 | G1 | 27 |
| 100 | 8 | G3 | 4 |
| 200 | 16 | G3 | 8 |
| 500 | 40 | G3 | 20 |

Table 2 indicates the internal volume of the different AFR reactors and the minimum and maximum flow rates to be used for a suitable mixing in those reactors.

TABLE 2
AFR Type and Reactor Flow Rates

| AFR module | Low Flow | G1 | G3 |
|---|---|---|---|
| Internal volume mL | 0.45 | 10 | 60 |
| Flow rate min mL/min | 2 | 30 | 400 |
| Flow rate max mL/min | 10 | 150 | 2000 |

In an embodiment, the passive mixing module may comprise a passive mixer with a T-junction or Y-junction. For example, the mixing step may be performed inside appropriately shaped junctions, such “T” or “Y”, using appropriate Reynolds number and turbulent mixing.

The passive mixing module may also be of the “manifold-type”. Such a passive mixing module device can comprise at least one in-line static mixer element connected after the T or Y junction. The outlet of the static mixer element is connected to a tubing that can be connected to one inlet of the cell culture vessel. The liquids may be pushed by means of peristaltic pumps, as peristaltic pumping prevents the liquids from being in contact with the pump body and therefore preserves the sterility. The tubing connected upstream the T or Y junction may be equipped with double-Y tubing in order to reduce the pulsations which are inherent to usual peristaltic pumps. Combining the split-channel tubing with the offset rollers of two stacked peristaltic pump heads reduces pulsation by merging a pulse from one channel with a trough from the other. By doing so, the flow of plasmid solution and the flow of transfection reagent solution are steady when entering the passive mixing zone. The passive mixing module may comprise aseptic connectors, such as genderless sterile disconnectors, as shown by 450.

In an embodiment, the passive mixing module may be offered as an aseptic module packaged or stored in containers. Any suitable aseptic treatment method may be used. Any suitable container or aseptic storage may be used. As a nonlimiting example, the passive mixing module may be made aseptic by gamma irradiation treatment and stored in pouches.

### Transfection Reagents

Any suitable transfection reagent may be used for transfecting cells in culture according to methods described herein. In an embodiment, the transfection reagent comprises a polymer such as polyethylenimine (PEI), calcium phosphate, DEAE-Dextran, polyarginine, dendrimers, and ionizable or cationic lipids, lipid-like lipidoids, or combinations of transfection reagents. In an embodiment, the transfection reagent comprises PEI. As a nonlimiting example, methods for transiently transfecting cells in culture may comprise using chemical transfection reagents. Nonlimiting examples of chemical transfection reagents include, for example PEI, calcium phosphate, DEAE-dextran, activated dendrimers, and cationic lipids. See, e.g. Kingston, R. E., et al., (2003).

In an embodiment, the transfection reagent comprises an inorganic transfection agent such as calcium phosphate. Though calcium phosphate (CaP) is particularly attractive due to its low cost and low cytotoxicity, CaP-mediated transfection is known to be hardly reproducible. A possible root cause of the variability is the rapid nucleic acid/CaP particle growth, which may be overcome using the continuous flow method described herein.

When CaP is used in methods as described herein, the nucleic acids may be blended with either the calcium source (such as a calcium salt) or the phosphate source. For example, where the plasmid(s) are blended with the phosphate source in one reservoir that is connected to the first inlet of the mixing fluidic device, and the calcium source is placed in a second reservoir connected to the second inlet of the mixing fluidic device. Upon mixing, the calcium phosphate is formed, which binds the plasmids and forms nucleic acid non-viral vector particles. Information on calcium phosphate-mediated transfection can be found in SPIZIZEN, J., REILLY, B. E., and EVANS, A. H. (1966). Microbial transformation and transfection. Annu. Rev. Microbid. 20, 371). As another example, the plasmid(s) may be blended first with the calcium source for some protocols, and then contacted with the phosphate source.

### Cell Culture Vessel or Bioreactor

Any suitable cell culture vessel or bioreactor may be used with methods according to embodiments described herein. In an embodiment, the bioreactor is a perfusion bioreactor. A nonlimiting example of a perfusion bioreactor is the Corning® Ascent® FBR system (Corning Incorporated, Corning, NY). In an embodiment, the bioreactor is a fixed-bed bioreactor. A nonlimiting example of a fixed-bed bioreactor is the iCELLis® bioreactor (Pall Corporation). In an embodiment, the bioreactor comprises packed-plain fibers, hollow fibers, packed or rolled fiber mesh, or packed-beads. In an embodiment, the bioreactor comprises a wave reactor.

In an embodiment, a cell culture vessel or bioreactor used with methods as described herein is configured for adherent cell culture.

In an embodiment, a cell culture vessel or bioreactor used with methods described herein is configured for suspension cell culture. For example, if a suspension reactor is used with methods according to embodiments described herein, the reactor may be equipped with a cell strainer or any other device that maintain the cells within the vessel during perfusion.

### EXAMPLES

Embodiments of the present disclosure are further described below with respect to certain exemplary and specific embodiments thereof, which are illustrative only and not intended to be limiting.

### Example 1

Example 1 illustrates the transfection of HEK 293T cell cultured in an Ascent® FBR system (Corning Incorporated, Corning, NY) small-scale perfusion bioreactor using polyplex prepared by means of a Low Flow AFR™ (Corning Incorporated, Corning, NY) passive mixer according to the methods described herein or prepared in batch.

In the experimental setup for the Low Flow AFR mixer, two syringe pumps were connected to two inlets of a Low Flow AFR mixer (Corning Incorporated, Corning, NY) having an internal volume of 0.48 ml, for which the outlet was connected to the inlet at the bottom of an Corning® Ascent® FBR system perfusion reactor cartridge (Corning Incorporated, Corning, NY). One syringe contained the plasmids mix and the other one contained the PEI solution.

### Cell Culture and Handling

All experiments were performed using adherent IEK293T cells cultivated in IMDM medium (Gibco) supplemented with 10% FBS (Gibco), 1× GlutaMAX™-I (Gibco) and 1000 U/mL Penicillin, 1000 ug/mL Streptomycin (Penicillin-Streptomycin, Gibco). Cells were maintained adherent on tissue culture treated surfaces at 37° C. and 5% CO₂. They were subcultured twice a week using 0.25% Trypsin with 0.1% EDTA (Gibco). Only cells from passages less than 10 were used.

Three days prior transfection, HEK293T cells were seeded at about 8300 cells/cm² in the reactor. Cells were added to the media stock bottle and perfused through the mesh reactor. Cell seeding was monitored and complete in around 3 h. A media exchange was performed one hour before transfection.

### DNA and PEI Solution Preparation

The helper-free system, consisting of three plasmid DNA (pAAV-MCS-GFP, pAAV-RC2 and pHelper) that allow the production rAAV virions by transient transfection, was purchased from Cell Biolab (#AAV-400). One of the plasmids carries a GFP sequence that allows to evaluate transfection efficacy. All plasmids were amplified in Escherichia coli and isolated using a Maxi prep plasmid QIAGEN kit.

Before transfection, the three plasmids were combined at a ratio 1:1:1 (mass) in unsupplemented IMDM in order to achieve 0.18 ug DNA/cm² of the mesh reactor and ~2.6 ug DNA/mL in the total volume. Typically, 144 ug of each plasmid were diluted in unsupplemented IMDM for a total volume of 6 mL. The solution was mixed briefly by vortex, kept at room temperature used within 15 min.

As transfection agent, PEIpro® (Polyplus®) was used at a ratio DNA:PEI of 1:2 (weight:weight) and N/P=15. PEIpro was mixed with unsupplemented IMDM. Typically, 864 uL PEIpro was diluted in unsupplemented IMDM for a total volume of 6 mL. The solution was mixed briefly by vortex, kept at room temperature, and used within 15 min.

### Cell Transfection

For batch transfection, an equal volume of DNA solution and PEIpro solution were mixed briefly by vortex. Typically, 6 mL of PEIpro solution was added to 6 mL of DNA solution and mixed by seven pulses of vortex. The solution was then kept for 10 min at room temperature with no agitation to allow polyplex formation. After 10 min, the polyplex solution was added to the media stock bottle of the reactor and allowed to perfuse the mesh reactor for cell transfection for 24 hours.

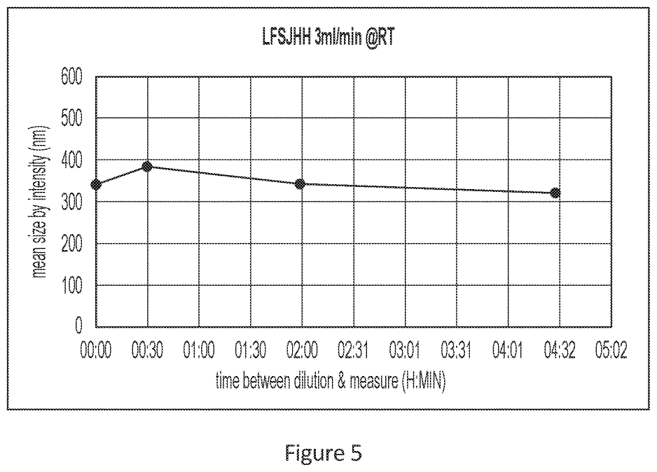

For AFR Low Flow transfection, sterile syringes were filled with the DNA solution and the PEIpro solution and connected to the AFR Low Flow reactor which itself is connected to the cell reactor (see scheme of the set up in FIG. 1). The mixing system was started and typically a total of 3 mL/min flow rate was used (1.5 mL/min for each syringe). An identical volume of polyplex solution was added to the mesh reactor compared to the batch transfection. The polyplex solution perfused the mesh reactor for 24 h. FIG. 5 is showing the hydrodynamic diameter of the polyplex particles determine by DLS as function of time once diluted in the culture media. The graph shows clearly that the size of polyplex particles is highly stable. The average hydrodynamic diameter of the polyplex is 300-400 nm.

At 24 h after transfection, a media exchange was performed for both batch and AFR Low Flow transfections.

### Polyplex Size Measurement

Polyplex size and surface charges was assessed using a Zetasizer Nano ZS. Polyplex solution from each transfection method were sampled either at the end of the 10 min incubation or right before entry in the mesh reactor system and diluted in complete IMDM, similarly to the dilution in the mesh reactor. For Polyplex size and surface charges evolution assessment during time, polyplex were kept in similar conditions as in the mesh reactor (37° C.) and measured at the appropriate time points.

### GFP Fluorescence Observation by Microscopy

At 72 h post-transfection, mesh reactors were opened, and some meshes at different area of the reactor were sampled for observation. The meshes were kept in complete IMDM in a petri dish and observation realized with an Olympus inverted fluorescent microscope. After observation, meshes were returned to the reactor for further analysis.

### GFP Fluorescence Quantification

At 72 hours post-transfection, the media was removed from the reactor and a PBS wash was performed. Cells were then dissociated from the mesh with a 45 min Accutase treatment and collected by centrifugation. Total cell amount for each sample was determined manually using a Malassez cell chamber. For each sample, GFP positive cells as well as mean fluorescence intensity were analyzed by flow cytometry using a BD Accuri C6 Plus system (BD Biosciences). Non-transfected cells were used as a negative control.

AAV2 Capsid (cAAV2) Titration

Total cells from the reactor were pelleted. Lysis buffer (2 mM Tris-HCl, 150 mM NaCl, 0.5% sodium deoxycholate, 495U benzonase) was added to the pellet and cell were lysed at 37° C. for 30 min. Cell lysate were centrifuged at 21000 g for 5 min to pellet cell debris.

AAV2 capsids titration was then performed using the AAV2 titration ELISA (#PRATV, PROGEN) following manufacturer recommendation.

Sample absorbance was measured using a BioTeck Gen5 and AAV2 titer determined according to the standard curve.

AAV2 Genome (gAAV2) Quantification

Total cells from the reactor were pelleted. Cell lysis and gAAV2 were quantified using the Takara AAVpro titration kit (#6233, Takara).

Briefly, cells were lysed using the lysis buffer provided in the kit. Cell lysate were treated with DNase followed by DNase inactivation and capsid lysis. AAV2 viral genomes were quantified by quantitative PCR using the reagents and methods provided and recommended by the Takara kit. The quantitative PCR was performed using a QuantStudio 6 Pro (Applied Biosystems).

### Data Analysis

Data were analyzed using the GraphPad Prism software.

### Polyplex Size and Surface Charges

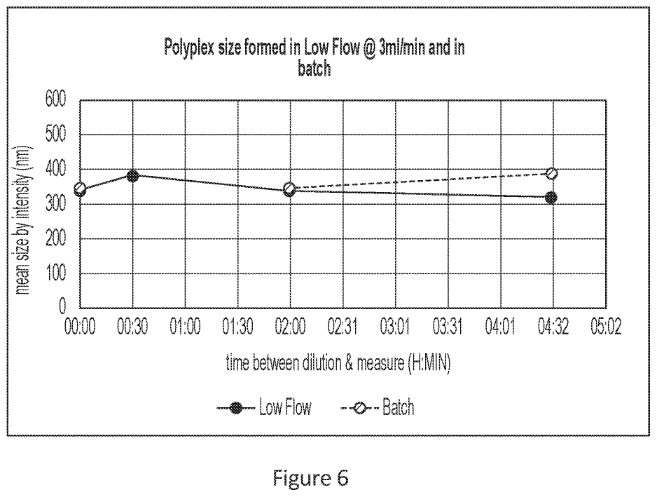

Polyplex size and surface charges was assessed using a Zetasizer Nano ZS. Polyplex solution from each transfection method were sampled either at the end of the 10 min incubation for batch or right before entry in the mesh reactor system for Low Flow preparation and diluted in complete IMDM, similar to the dilution in the mesh reactor. Polyplex size measurement and size evolution assessment during time (at 37° C.) are presented in FIG. 6 for batch and Low Flow preparation methods. Sizes are very close around 350 nm for both preparation methods and there is no significant evolution of size with time.

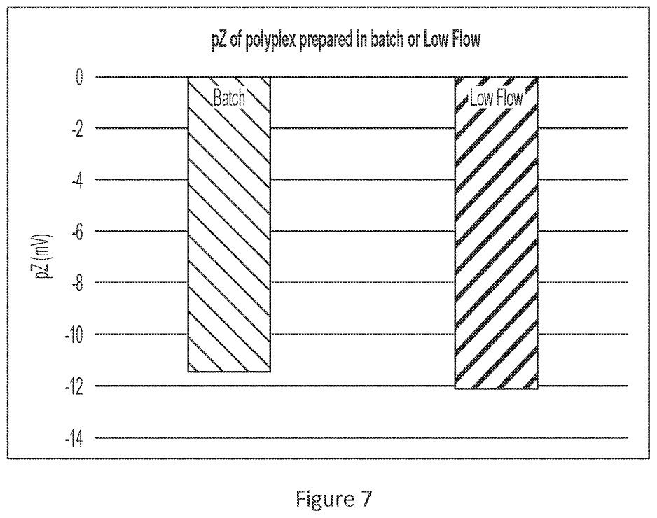

Potential zeta measurement of complexes formed with both preparation methods are presented in FIG. 7. There is no significant difference between two preparation methods with potential zeta being close to −12 mV.

### Comparison of Batch Transfection Method and AFR Low Flow Transfection Method

For both methods, identical PEIpro solution and DNA solution with the helper free system for AAV2 production were prepared with a DNA:PEI ration of 1:2.

The solutions were mixed briefly and allowed to stand for 10 min prior addition to the cell reactor for the Batch method. In the AFR Low Flow method, both solutions were connected to the AFR reactor in which they were mixed and then directly added to the cell reactor (direct connection).

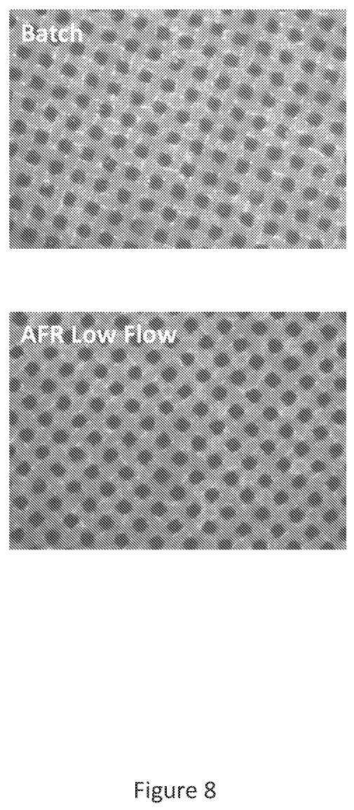

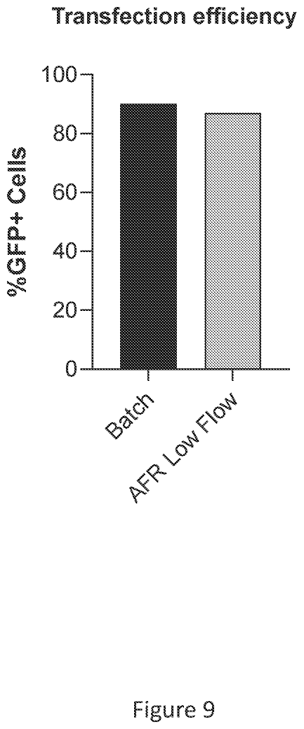

At 72 h post-transfection, GFP expression was observed and analyzed as well as AAV2 titer determined. In both methods, cells are homogenously adherent on the mesh and expressing a similar lever of GFP (FIG. 8). A flow cytometry analysis indicated that in both methods, a similar quantity of GFP positive cells were present in each sample suggesting a similar level of transfection for both the Batch and the AFR Low Flow methods (FIG. 9).

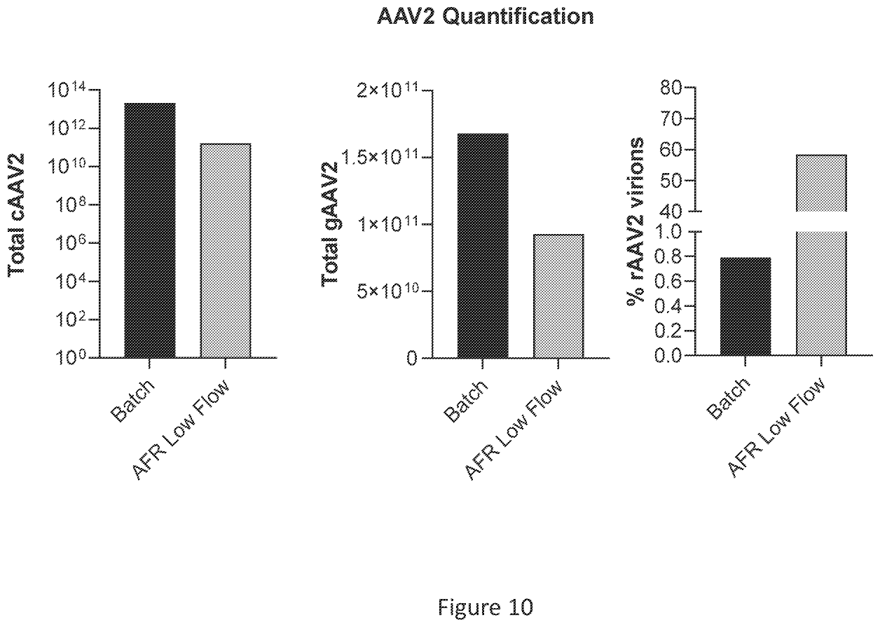

The cells were then lysed and treated to characterize the quality of the recombinant adeno-associated virus (rAAV2) produced. The quantify of total AAV2 capsid (cAAV2) produced was analyzed on one side and specifically the quantify of AAV2 viral genomes (gAAV2) on the other side. AAV2 capsids produced were about 20-fold higher in the Batch sample compared to the AFR Low Flow sample. However, AAV2 viral genomes produced were only 1.4-fold higher in the Batch sample compared to the AFR Low Flow method, indicating that both methods led to a similar amount of rAAV2 virion (full capsids) production (FIG. 10).

Moreover, the data indicate that the level of rAAV2 virion is about 8.57% with the AFR Low Flow method, while it is only about 0.8% with the Batch method. Such a strong increase of rAAV2 virions will then facilitate downstream rAAV2 purification methods.

Altogether, the data indicate that both the batch and the AFR Low Flow transfection method lead to a similar level of cell transfection and allow the production of AAV2 capsids and virions. However, although a similar level of transfection and rAAV2 virion are obtained, the level of rAAV2 virions is significantly higher with the AFR Low Flow method compared to the Batch method, facilitating downstream virion purification. In addition, the process described in the methods of the present disclosure made in flow can be readily scaled-up, whereas the batch process cannot. That clearly show the advantage of the method of the present disclosure in terms of industrialization.

### Example 2

Example 2 illustrates a comparison between T junction, AFR, and Batch methods, particularly the comparison of polyplex size and surface charges between the Batch method, AFR Low Flow method, and the T-junction method.

### Experimental Set-Up

The system set up, cell culture, and polyplex preparation were handled as described earlier with respect to Example 1 for Batch and AFR Low Flow conditions.

For the T-junction method, the AFR Low Flow reactor was replaced by a classical T-junction with no other parameter change. In this setup, the T-junction plays the role of passive mixer (T-mixer).

### Polyplex Size and Surface Charges

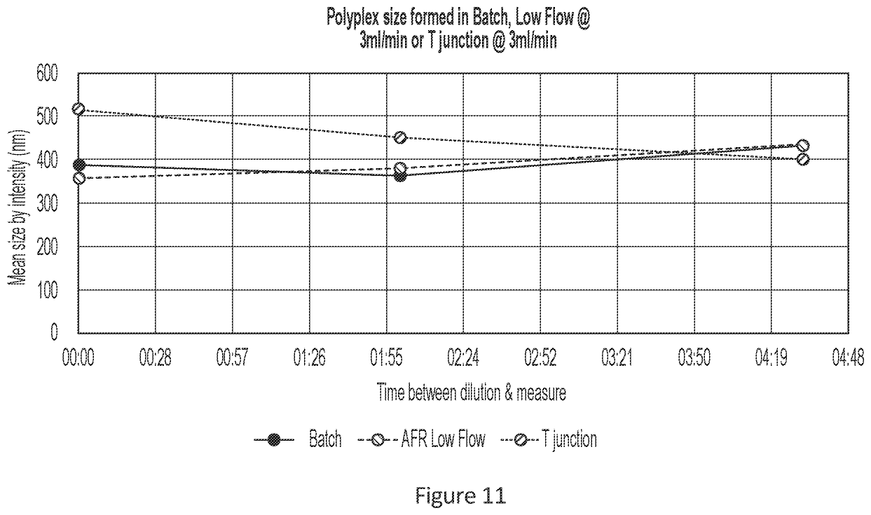

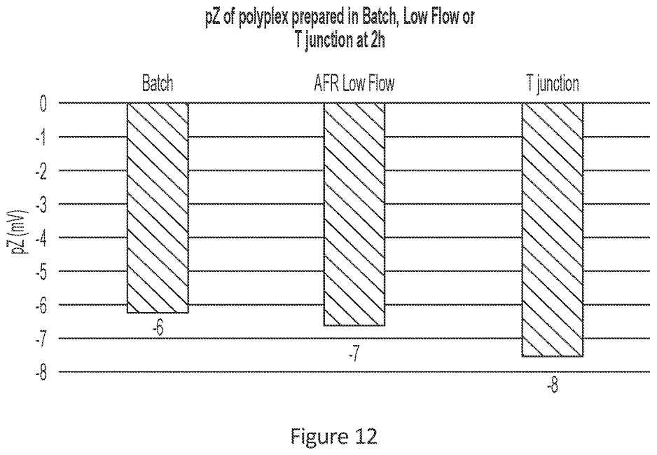

Polyplex size and surface charges were assessed using a Zetasizer Nano ZS and handled as described earlier. Although polyplex size was slightly higher for the T junction sample initially, its size decreased with time and stabilized at a similar level as the batch and AFR samples at about 3 hours. As previously determined, batch and AFR samples are of similar size and stable over time. Potential zeta measurement of polyplex generated in all three methods are similar. Polyplex size and potential zeta are presented in FIG. 11 and FIG. 12.

### Comparison of Batch Transfection Method, AFR Low Flow and a T Junction Transfection Method

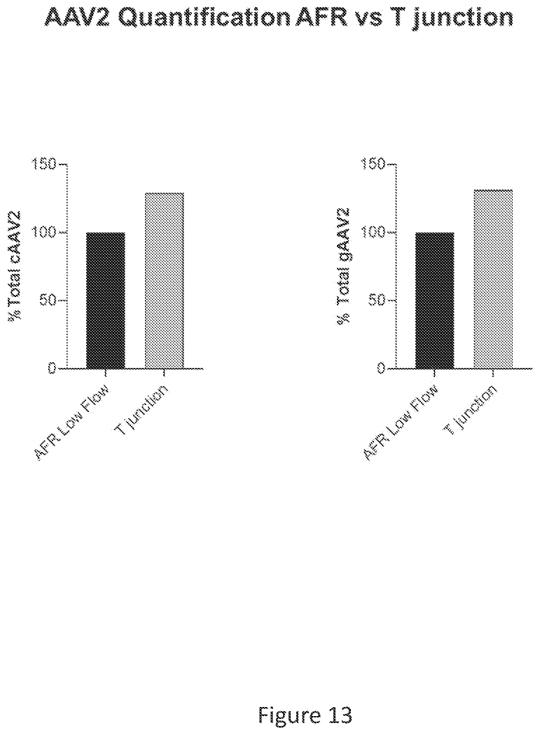

As described earlier, AAV2 production was assessed at 72 hours after transfection both for total viral genome (gAAV2) and total capsid (cAAV2) production. These data are presented in FIG. 13. Interestingly, the T junction method led to a slightly higher capsid production (cAAV2) and viral genomes (gAAV2) than the AFR method.

Altogether, these data indicate that in this set up and experimental conditions, polyplex mixing by an AFR reactor or T junction lead to a similar cAAV2 ang gAAV2 production.

### Scalable Batch Preparation

Embodiments of this disclosure includes scalable systems and methods for performing the transfection step in biomanufacturing processes, including, for example, AAV production. The systems and methods for transfection can be scaled from small scale (e.g., a process development bioreactor) to large scale (e.g., a pilot and/or production scale bioreactor). The ability to directly scale up the systems and methods enables a stable and well-defined manufacturing process. The disclosed systems and methods not only enable scale up, but also allow for maintenance of critical process parameters such as DNA concentration of the transfection solution, efficiency and speed of mixing PEI and DNA solutions, the time the transfection solution is incubated for formation of complexes (e.g., PEI-DNA nanoparticles) of appropriate size before being added to the cells, and kinetics of the transfection mixture addition.

To successfully scale up a transfection step from a 2D laboratory scale process to a 3D bioreactor production scale process, various process parameters have to be maintained constant. These parameters include: (i) concentration of PEI in reagent solution; (ii) concentration of DNA in reagent solution; (iii) ratio of molar units of PEI nitrogen atoms to units of DNA phosphate atoms; (iv) reagents mixing time; (v) PEI-DNA nano particle complex formation time, i.e. final nanoparticle size; and (vi) final concentration of DNA in cell culture system, both μg/cell and μg/ml of media. The systems and methods of this disclosure enable a scalable process of the transfection reagent preparation step for process development and production scales.

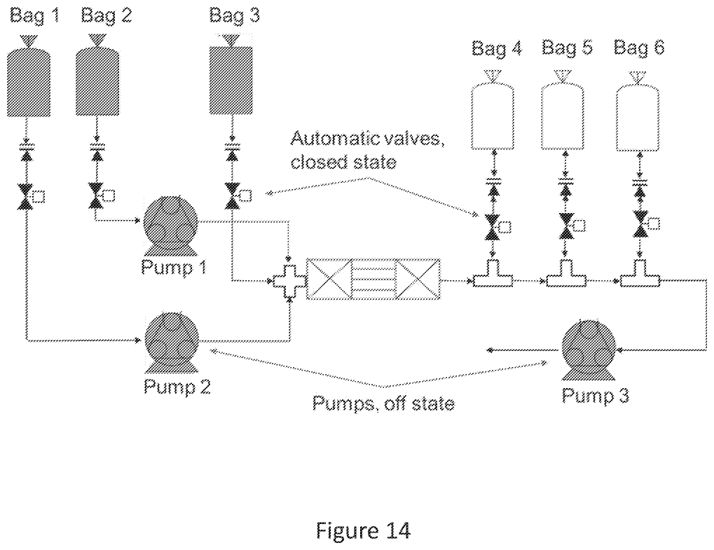

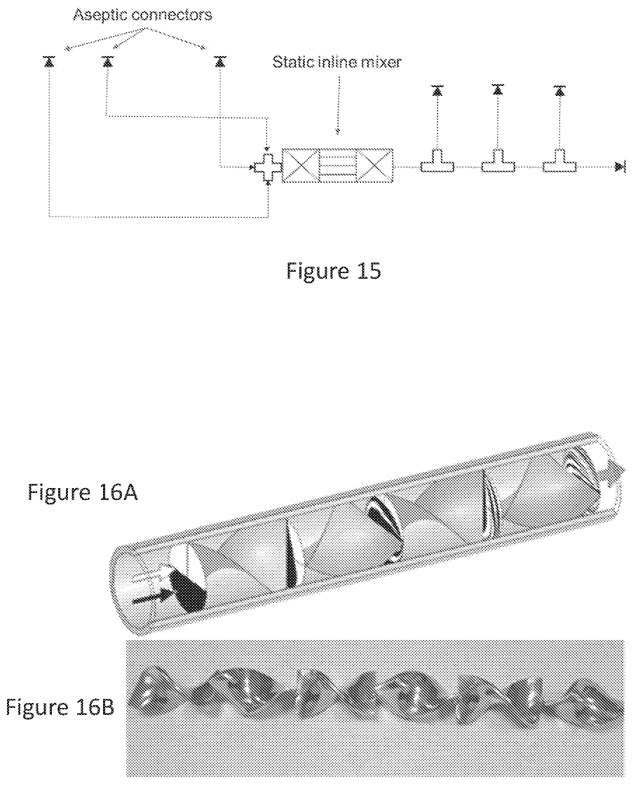

An example of the system is shown in FIG. 14. According to embodiments, the system can include a single use set of sterile bioprocess consumables, including single use bags and single use transfer tubing with appropriate aseptic connectors as shown in FIG. 15. The example embodiment in FIG. 15 shows a consumable designed for a system with three single-use bags for incubation of PEI-DNA complex. The mixer can be any of various mixers for preparing a transfection complex, including those discussed herein. A further example of a static mixer integrated into single use consumable set is show in FIGS. 16A and 16B. An inline single use static mixer can be integrated into the tubing of the single use consumable set. The system in FIG. 14 further includes a set of peristaltic pumps and automatic open/close valves. The operation sequence of the peristaltic pumps and automatic valves can be preprogrammed to deliver conditions of transfection complex formation that are scalable from process development scale to production scale.

According to embodiments, original reagent solutions of PEI and DNA are prepared offline and transferred into single use bags 1 and 2. Bag 3 contains predefined amount of cell culture media that will be used at the end of the process to flush the system thus increasing efficiency of PEI/DNA complex utilization. Bags 4, 5, and 6 are used for incubation of PEI-DNA complex during nanoparticle complex formation and particle size growth. Peristaltic pumps 1 and 2 are used to deliver the original solution reagents from bag 1 and bag 2 to the static inline mixer. During the pump's operation, reagent solutions become uniformly mixed at the exit of the inline mixer. Flow of mixed solution is automatically directed by preprogrammed automatic valves into bags 6, 5 and 4, where it would be incubated for predefined amount of time to form nanoparticle complexes of defined size. According to embodiments, the filling of bags 6, 5, and 4 can be done consecutively, such that bag 6 is filled first, followed by bag 5, then bag 4.

After a predefined incubation time, pump 3 will transfer the PEI-DNA nanoparticles into main media conditioning vessel in consecutive manner from bag 6, 5, and 4 correspondingly. Then, all system components can be washed by cell culture media to increase the recovery of PEI-DNA nanoparticles from single-use consumables.

Table 3 shows key process parameters for scale up of the transfection reagent during the preparation step, according to embodiments. As can be seen from Table 3, marked key parameters critical to the process of PEI-DNA nanoparticle complex formation are maintained constant upon scale up.

| | Process development scale | | | Pilot scale | | | Production scale | | | |
|---|---|---|---|---|---|---|---|---|---|---|
| FBR size, m² | 1 | 2.5 | 5 | 20 | 50 | 100 | 200 | 500 | 1000 | |
| Volume of PEI | 0.03 | 0.075 | 0.15 | 0.6 | 1.5 | 3 | 6 | 15 | 30 | |
| solution, L | | | | | | | | | | |
| Volume of DNA | 0.03 | 0.075 | 0.15 | 0.6 | 1.5 | 3 | 6 | 15 | 30 | * |
| solution, L | | | | | | | | | | |
| Volume of | 0.06 | 0.15 | 0.3 | 1.2 | 3 | 6 | 12 | 30 | 60 | |
| transfection | | | | | | | | | | |
| mixture solution, L | | | | | | | | | | |
| Solution mixing | 0.015 | 0.015 | 0.015 | 0.3 | 0.3 | 0.3 | 3 | 3 | 3 | * |
| flow rate | | | | | | | | | | |
| Number of | 1 | 3 | 5 | 1 | 3 | 5 | 1 | 3 | 5 | |
| incubation Bags | | | | | | | | | | |
| Volume of | 0.06 | 0.05 | 0.06 | 1.2 | 1 | 1.2 | 12 | 10 | 12 | |
| transfection mixture | | | | | | | | | | |
| per bag | | | | | | | | | | |
| Time to fill one | 2 | 1.7 | 2.0 | 2.0 | 1.7 | 2.0 | 2.0 | 1.7 | 2.0 | |
| incubation bag | | | | | | | | | | |
| Time to mix | 2 | 5 | 10 | 2 | 5 | 10 | 2 | 5 | 10 | * |
| tansfection reagent | | | | | | | | | | |
| Flow rate of | 0.03 | 0.03 | 0.03 | 0.60 | 0.60 | 0.60 | 6.00 | 6.00 | 6.00 | |
| transferring | | | | | | | | | | |
| transfection mixture | | | | | | | | | | |
| into the MCV | | | | | | | | | | |
| Delay after mixing | 8.00 | 5.00 | 0.00 | 8.00 | 5.00 | 0.00 | 8.00 | 5.00 | 0.00 | |
| all transfection | | | | | | | | | | |
| reagent and | | | | | | | | | | |
| beginning of | | | | | | | | | | |
| transfer of | | | | | | | | | | |
| transfection mixture | | | | | | | | | | |
| into the MCV | | | | | | | | | | |
| Transfection | 10.00 | 10.00 | 10.00 | 10.00 | 10.00 | 10.00 | 10.00 | 10.00 | 10.00 | * |
| mixture total | | | | | | | | | | |
| incubation time | | | | | | | | | | |
| Time to transfer | 2 | 5 | 10 | 2 | 5 | 10 | 2 | 5 | 10 | * |
| transfection mixture | | | | | | | | | | |
| to the MCV | | | | | | | | | | |

It will be appreciated that the various disclosed embodiments may involve particular features, elements, or steps that are described in connection with that particular embodiment. It will also be appreciated that a particular feature, element, or step, although described in relation to one particular embodiment, may be interchanged or combined with alternate embodiments in various non-illustrated combinations or permutations.

It is also to be understood that, as used herein the terms “the,” “a,” or “an,” mean “at least one,” and should not be limited to “only one” unless explicitly indicated to the contrary. Thus, for example, reference to “an opening” includes examples having two or more such “openings” unless the context clearly indicates otherwise.

All scientific and technical terms used herein have meanings commonly used in the art unless otherwise specified. The definitions provided herein are to facilitate understanding of certain terms used frequently herein and are not meant to limit the scope of the present disclosure.

As used herein, “have,” “having,” “include,” “including,” “comprise,” “comprising,” or the like are used in their open-ended sense, and generally mean “including, but not limited to.”

Ranges may be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All numerical values expressed herein are to be interpreted as including “about,” whether or not so stated, unless expressly indicated otherwise. It is further understood, however, that each numerical value recited is precisely contemplated as well, regardless of whether it is expressed as “about” that value. Thus, “a dimension less than 10 mm” and “a dimension less than about 10 mm” both include embodiments of “a dimension less than about 10 mm” as well as “a dimension less than 10 mm.”

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method comprising A+B+C include embodiments where a method consists of A+B+C, and embodiments where a method consists essentially of A+B+C.

Although multiple embodiments of the present disclosure have been described in the Detailed Description, it should be understood that the disclosure is not limited to the disclosed embodiments, but is capable of numerous rearrangements, modifications and substitutions without departing from the disclosure as set forth and defined by the following claims.

1. A method for preparing a transfection reagent comprising:
providing a plurality of reagent components for mixing together to form the transfection reagent;
mixing the plurality of reagent components in a passive mixing module comprising a plurality of inlets and an outlet, the plurality of inlets being for receiving the plurality of reagent components and the outlet being for outputting a mixture of the plurality of reagent components;
providing a plurality of incubation containers fluidly connected to the outlet and configured for receiving and holding the mixture;
filling one or more of the plurality of incubation containers with at least a portion the mixture from the outlet, the filling of the one or more of the plurality of incubation containers occurring consecutively;
holding the at least a portion of the mixture in one or more of the plurality of incubation containers for a predetermined incubation time to form the transfection reagent.

2. The method of claim 1, further comprising:
removing the at least a portion of the mixture from the one or more of the plurality of incubation containers after the predetermined incubation time.

3. The method of claim 2, wherein the removing is performed for each of the one or more of the plurality of incubation containers consecutively.

4. The method of claim 3, wherein the removing is performed consecutively based on when the predetermined incubation time for each of the one or more plurality of incubation containers is reached.

5. The method of claim 1, wherein the predetermined incubation time is based on a desired size of nanoparticle complexes in the transfection reagent.

6. The method of claim 1, further comprising, after the transfection reagent is formed, washing at least one of the passive mixing module, the plurality of incubation containers, and any tubing, valves, or pumps with a liquid media to remove any remaining transfection reagent.

7. (canceled)

8. The method of claim 1, wherein the passive mixing module comprises a microfluidic mixing device.

9. The method of claim 8, wherein the microfluidic mixing device comprises a plurality of mixing elements.

10. The method of claim 9, wherein the plurality of mixing elements comprises heart-shaped mixing elements.

11. The method of claim 8, wherein the microfluidic mixing device comprises a continuous microfluidic flow reactor.

12. The method of claim 11, wherein the continuous microfluidic flow reactor is configured for scale-up from lab bench to industrial manufacturing.

13. The method of claim 1, wherein the transfection reagent comprises polyethylenimine (PEI), calcium phosphate, DEAE-Dextran, polyarginine, dendrimers, ionizable or cationic lipids, lipid-like lipidoids, plasmids or combinations thereof.

14. The method of claim 1, wherein the method further comprises transfecting cells using the transfection reagent.

15. The method of claim 1, further comprising flowing the transfection reagent to a culture vessel.

16. The method of claim 15, wherein the culture vessel comprises a bioreactor.

17. The method of claim 15, wherein the culture vessel comprises a perfusion bioreactor.

18. (canceled)

19. The method of claim 15, wherein the culture vessel comprises cells to be transfected.

20. The method of claim 15, wherein the culture vessel comprises culture medium.

21. The method of claim 1, wherein the mixing module comprises a disposable mixing device.

22. The method of claim 1, wherein the mixing module comprises a reusable mixing device.
